(19) United States
(12) Patent Application Publication
(10) Pub. No.: US 2010/0226932 A1
(43) Pub. Date: Sep. 9, 2010

(54) Adjuvant and Vaccine Compositions

(75) Inventors: Gail Smith, Gaithersburg, MD (US); Dinesh B. Shenoy, Karnataka (IN); Robert W. Lee, Boyertown, PA (US)

Correspondence Address: COOLEY LLP;ATTN: Patent Group, Suite 1100, 777 - 6th Street, NW, WASHINGTON, DC, 20001, US

(73) Assignee: NOVAVAX, INC., Rockville, MD (US)

(21) Appl. No.: 12/280,099

(22) PCT Filed: Feb. 22, 2007
(86) PCT No.: PCT/US2007/004470

§ 371 Date: May 24, 2010

Related U.S. Application Data
(60) Provisional application No. 60/775,346, filed on Feb. 22, 2006.
(60) Provisional application No. 60/861,245, filed on Nov. 28, 2006.

(51) Int. Cl. A61K 39/00; A61P 37/04

(52) U.S. Cl. 424/184.1

(57) ABSTRACT

Abstract Compositions comprising an emulsion and aluminum salt nano-/micro-particles surface stabilized with at least one surfactant are useful as immunological adjuvants. The emulsion of these compositions comprises at least one oil; at least one surfactant; a plurality of surfactant vesicles; optionally at least one sterol; and an aqueous phase. The present invention also provides vaccines comprising one or more antigens combined with the emulsion and surface stabilized aluminum salt particles of the present invention, or one or more antigens combined with non-ionic surfactant vesicles.

This application claims priority to provisional applications 60/775,346, filed Feb. 22, 2006 and 60/861,245, filed Nov. 28, 2006, both of which are incorporated by reference in their entirety for all proposes.

### TECHNICAL FIELD

This invention relates generally to compositions useful as immunological adjuvants and compositions useful for enhancing an immune response in a subject. In particular, this invention is directed to a composition comprising an emulsion, surfactant vesicles and surface stabilized aluminum salt micro-/nano-particles, as well as methods of preparing such compositions, and methods of treatment. In addition, this invention is directed to non-ionic surfactant vesicles, compositions, methods of treatment, and methods of preparing compositions comprising a non-ionic surfactant vesicles with specific antigens.

### BACKGROUND OF THE INVENTION

Immunological adjuvants are the component(s) of a vaccine which augment the immune response to the antigen. Immunological adjuvants function by attracting macrophages to the antigen and then presenting the antigen to the regional lymph nodes to initiate an effective antigenic response. Adjuvants may also act as carriers themselves for the antigen. Many of the known immunological adjuvants produce undesirable reactions in humans such as inflammation at the site of injection. These side effects can limit the use of such adjuvants in humans, and have led to the search for alternative immunological adjuvants.

Immunological adjuvants included mineral compounds, oil emulsions, bacterial products, liposomes, immunostimulating complexes, and other adjuvants such as squalene. These adjuvants have substantially different chemical properties, and have in common only their ability to enhance the immune response. In addition, these adjuvants are also highly variable in their effect on the immune system (including adverse side effects). For example, bacterial products can be extremely toxic, and oil emulsions (e.g. Freund's adjuvants) can produce autoimmune responses.

Aluminum compounds are the most widely used adjuvants in human and veterinary vaccines. Aluminum adjuvant compounds include aluminum salts such as aluminum phosphate (AlPO₄) and aluminum hydroxide (Al(OH)₃) compounds, typically in the form of gels, and are generically referred to in the field of vaccine adjuvants as “alum”. Aluminum hydroxide is a poorly crystalline aluminum oxyhydroxide having the structure of the mineral boehmite. Aluminum hydroxide gels have a pI of 11. Aluminum phosphate is an amorphous aluminum hydroxyphosphate, and depending upon how the aluminum phosphate gels are prepared, pI values can range from 5-7. Thus, negatively charged species (e.g., negatively charged antigens) can absorb onto aluminum hydroxide gels at neutral pH, whereas positively charged species (e.g., positively charged antigens possibly charged proteins) can absorb onto aluminum phosphate gels at neutral pH. It is believed that alum adjuvants provide a depot of antigen at the site of administration (e.g., injection), thereby providing a gradual and continuous release of antigen to stimulate antibody production, and appear to primarily stimulate IL-4 and T-helper-2 cells, and enhance IgG1 and IgE production.

Aluminum hydroxide has been determined to be a more potent adjuvant than aluminum phosphate. However, it can be difficult to absorb positively charged antigens onto aluminum hydroxide gels because aluminum hydroxide also has a positive charge at neutral pH. Furthermore, alum adjuvants can cause mild local reactions at the site of injection, and can remain at the site of a subcutaneous injection for up to one year after injection. In addition, alum gels also cannot be frozen or easily lyophilized because both processes caused the gel to collapse, resulting in gross aggregation and precipitation. Thus, even though alum adjuvants are useful, it would be desirable to reduce the amount of alum needed, and/or provide alternative adjuvants without these drawbacks.

Emulsion adjuvants include water-in-oil emulsions (e.g., Freund's adjuvants) and oil-in-water emulsions (e.g., MF-59). Emulsion adjuvants include an immunogenic component, for example squalene (MF-59) or mannide oleate (Incomplete Freund's Adjuvants), which can induce e.g., elevated humoral response, increased T cell proliferation, cytotoxic lymphocytes, and cell-mediated immunity. Emulsion adjuvants are unstable upon freezing, and exposure to pH extremes can hydrolyze the surfactant components. In addition, some components are susceptible to oxidation in the presence of oxygen, peroxide, or metals.

Liposomal or vesicular adjuvants (including paucilamellar lipid vesicles) have lipophilic bilayer domains and an aqueous milieu which can be used to encapsulate and transport a variety of materials, for example an antigen. Paucilamellar vesicles (e.g., those described in U.S. Pat. No. 6,387,373) can be prepared e.g., by mixing, under high pressure or shear conditions, a lipid phase comprising a non-phospholipid material (e.g., an amphiphile surfactant; see U.S. Pat. Nos. 4,217,344; 4,917,951; and 4,911,928), optionally a sterol, and any water-immiscible oily material to be encapsulated in the vesicles (i.e., an oil such as squalene oil and an oil-soluble or oil-suspended antigen); and an aqueous phase comprising e.g., water, saline, buffer or any other aqueous solution used to hydrate the lipids. Liposomal or vesicular adjuvants are believed to promote contact of the antigen with immune cells (e.g., by fusion of the vesicle to the immune cell membrane), and preferentially stimulate the Th1 sub-population of T-helper cells. Liposomal or vesicular adjuvants are incompatible with most organic solvents and some detergents, and are osmotically sensitive.

Commercially available amphiphile surfactants include, for example, the BRIJ™ family of polyoxyethylene fatty ethers, the SPAN™ sorbitan fatty acid esters, and the TWEEN™ polyoxyethylene derivatives of sorbitan fatty acid esters, all available from ICI Americas, Inc. of Wilmington, Del. Paucilamellar vesicles containing such amphiphiles provide a high carrying capacity for water-soluble and water immiscible substances. The high capacity for water immiscible substances represents a unique advantage over classical phospholipid multilamellar liposomes.

Paucilamellar lipid vesicles may include a wide variety of phospholipids and non-phospholipid surfactants as their primary structural material. Paucilamellar lipid vesicles are substantially spherical structures made of materials having high lipid content, preferably from non-phospholipid materials, which are organized in the form of lipid bilayers. The two to ten peripheral bilayers encapsulate an aqueous volume which is interspersed between the lipid bilayers and may also be encapsulated in the amorphous central cavity. Alternatively, the amorphous central cavity may be substantially filled with a water immiscible material, such as an oil or wax. Paucilamellar lipid vesicles have advantages as transport vehicles because the large unstructured central cavity is easily adaptable for transport of large quantities of aqueous or oleaginous materials. Novasomes® are paucilamellar lipid vesicles ranging from about 100 nm to about 500 mm. They comprise BRIJ™ 72, cholesterol, oleic acid and squalene. Novasomes® have been shown to be an effective adjuvant for influenza antigens (see, U.S. Pat. Nos. 5,629,021 and 6,387,373).

Non-ionic surfactant vesicles (niosomes) prepared from a non-ionic surfactant, cholesterol and dicetyl phosphate are known in the art and have been extensively used in the cosmetic industry. Niosomes appear to be multilamellar surfactant structures. Relatively insoluble compounds, such as the chemotherapeutics currently formulated in liposomes, could be delivered in these synthetic, nonionic surfactant vehicles. A method for producing niosomes involves drying the lipid to a thin film from organic solvent, and then hydrating this film with the aqueous solvent of choice. The resulting multilamellar vesicles can be further processed by sonication, extrusion, or other treatments to optimize drug entrapment. Other methods, such as injection of lipids in water-miscible or water-immiscible solvents into an aqueous solution, detergent dialysis, or reverse-phase evaporation are also available.

The above classes of adjuvants have different chemical properties, and function by different biochemical mechanisms. Thus, no single adjuvant may be effective for all antigens. Accordingly, there is a need for a more universal adjuvant which minimizes the drawbacks of known adjuvants, and would be useful with any adjuvant. In addition, there is a need for adjuvants having a stronger immunostimulatory effect.

### BRIEF SUMMARY OF THE INVENTION

In one embodiment, the present invention relates to a composition comprising an emulsion and aluminum salt nano-/micro-particles surface stabilized with at least one surfactant, wherein the emulsion comprises at least one oil, at least one surfactant, a plurality of surfactant vesicles, optionally at least one sterol, and an aqueous phase.

In another embodiment, the present invention relates to a vaccine comprising at least one antigen, and emulsion, and aluminum salt nano-/micro-particles surface stabilized with at least one surfactant, wherein the emulsion comprises at least one oil, at least one surfactant, a plurality of surfactant vesicles, optionally at least one sterol, and an aqueous phase.

In a further embodiment, the present invention relates to an immunogenic composition comprising a non-ionic surfactant vesicle and a selected antigen entrapped in, adsorbed to, or in admixture with said vesicle.

In another embodiment, the present invention relates to a method of immunization which comprises administering to a vertebrate subject a therapeutically effective amount of a selected antigen combined with a composition comprising an emulsion and aluminum salt nano-/micro-particles surface stabilized with at least one surfactant, wherein the emulsion comprises at least one oil, at least one surfactant, a plurality of surfactant vesicles, optionally at least one sterol, and an aqueous phase.

In yet a further embodiment, the present invention relates to a method of immunization which comprises administering to a vertebrate subject (a) a non-ionic surfactant vesicle, and (b) a therapeutically effective amount of a selected antigen entrapped in, or adsorbed to, said vesicle. In still a further embodiment, said method comprises a method of immunization wherein said vesicle and the antigen are co-administered, regardless of entrapment or absorption.

In still further embodiments, the invention relates to a method of making a composition comprising combining non-ionic surfactant vesicles with a selected antigen entrapped in, or adsorbed to, said vesicle. In another embodiment, the method of making comprises an admixture of vesicles and antigens, regardless of entrapment or absorption. In an additional embodiment, the non-ionic surfactant vesicle is a Novasome® and/or niosome. In another embodiment, said method of making a composition comprises mixing a vesicle and an antigen, regardless of entrapment or absorption.

In another embodiment, the present invention relates to a composition prepared by a process comprising (a) combining at least one oil, an aqueous phase, aluminum salt particles, at least one surfactant, and optionally at least one sterol, and (b) mixing the combination of step (a) under shear mixing conditions, whereby the at least one oil, the aqueous phase, at least one surfactant, and optionally at least one sterol form an emulsion and a plurality of surfactant vesicles, and the aluminum salt particles are reduced in size to form aluminum salt nano-/micro-particles surface stabilized with at least one surfactant.

In another embodiment, the present invention relates to an adjuvanted vaccine prepared by a process comprising (a) combining at least one oil, an aqueous phase, aluminum salt particles, at least one surfactant, and optionally at least one sterol, (b) mixing the combination of step (a) under shear mixing conditions, whereby the at least one oil, the aqueous phase, at least one surfactant, and optionally at least one sterol form an emulsion and a plurality of surfactant vesicles, and the aluminum salt particles are reduced in size to form aluminum salt nano-/micro-particles surface stabilized with at least one surfactant, and (c) adding an immunologically effective amount of at least one antigen.

In another embodiment, the present invention relates to a process comprising (a) combining at least one oil, an aqueous phase, aluminum salt particles, at least one surfactant, and optionally at least one sterol, and (b) mixing the combination of step (a) under shear mixing conditions, whereby the at least one oil, the aqueous phase, at least one surfactant, and optionally at least one sterol form an emulsion and a plurality of surfactant vesicles, and the aluminum salt particles are reduced in size to form aluminum salt nano-/micro-particles surface stabilized with at least one surfactant.

In another embodiment, the present invention relates to a process comprising (a) combining at least one oil, an aqueous phase, aluminum salt particles, at least one surfactant, and optionally at least one sterol, (b) mixing the combination of step (a) under shear mixing conditions, whereby at least one oil, the aqueous phase, at least one surfactant, and optionally at least one sterol form an emulsion and a plurality of surfactant vesicles, and the aluminum salt particles are reduced in size to form aluminum salt nano-/micro-particles surface stabilized with at least one surfactant, and (c) adding an immunologically effective amount of at least one antigen.

### DETAILED DESCRIPTION OF THE INVENTION

In one embodiment, the compositions of the present invention comprise an emulsion and aluminum salt nano-/micro-particles surface stabilized with at least one surfactant, wherein the emulsion comprises at least one oil, at least one surfactant, a plurality of surfactant vesicles, optionally at least one sterol, and an aqueous phase.

The compositions of the present invention are useful as immunological adjuvants. As used herein the term “adjuvant” refers to a compound that, when used in combination with one or more specific immunogens (e.g., antigens) in a formulation, augments or otherwise alters or modifies the resultant immune response. Modification of the immune response includes intensification or broadening the specificity of either or both antibody and cellular immune responses. Modification of the immune response can also mean decreasing or suppressing certain antigen-specific immune responses.

The compositions of the present invention comprise one or more oils in the form of an emulsion. Non-limiting examples of suitable oils for the compositions of the present invention include vegetable oils, nut oils, fish oils, lard oil, mineral oils, water insoluble vitamins such as vitamin E and mixtures thereof. Specific non-limiting examples of oils that may be used include almond oil (sweet), apricot seed oil, borage oil, canola oil, coconut oil, corn oil, cotton seed oil, fish oil, jojoba bean oil, lard oil, linseed oil (boiled), Macadamia nut oil, triglyceride oil such as medium chain triglycerides, mineral oil, petrolatum, olive oil, peanut oil, safflower oil, sesame oil, soybean oil, squalene, squalane, sunflower seed oil, tricaprylin (1,2,3-trioctanoyl glycerol), wheat germ oil, rapeseed oil, avocado oil, flavor oils, and mixtures thereof.

The oil component of the adjuvant compositions of the present invention can include one or more immunogenic oils, one or more non-immunogenic oils, or a mixture of immunogenic and non-immunogenic oils. Immunogenic oils are oils which themselves induce an immunogenic response. Non-limiting examples of immunogenic oils include squalane and squalene. Non-immunogenic oils are oils which by themselves do not induce an immunogenic response, but in the form of an emulsion comprising vesicles can function as part of an immunological adjuvant, as described in U.S. Pat. No. 6,387,373, herein incorporated by reference. In one embodiment, the oil component of the adjuvant compositions of the present invention is soybean oil.

In another embodiment, the compositions of the present invention comprise about 0.5-40.0 wt. % of one or more oils (i.e., the wt. % of the total amount of oil in the composition, based on the total weight of the composition). In another embodiment, the compositions of the present invention comprise about 0.5-30.0 wt. %, or about 0.5-20.0 wt. %, or about 0.5-10.0 wt. %, or about 0.5-9.0 wt. %, or about 0.5-8.0 wt. %, or about 0.5-7.0 wt. %, or about 0.5-6.0 wt. %, or about 0.5-5.0 wt. % or about 0.5-4.0 wt. % of one or more oils. In another embodiment, the compositions of the present invention comprise about 1.0-10.0 wt. %, or about 1.5-10.0 wt. %, or about 2.0-10.0 wt. %, or about 2.5-10.0 wt. %, or about 3.0-10.0 wt. %, or about 3.0-9.5 wt. %, or about 3.0-9.0 wt. %, or about 3.0-8.5 wt. %, or about 3.0-8.0 wt. %, or about 3.0-7.5 wt. %, or about 3.0-7.0 wt. %, or about 3.0-6.5 wt. %, or about 3.0-6.0 wt. % of one or more oils. The term “about” in regard to the weight range of the oil component refers to both the upper and lower limit specified. Thus, about 1.0-10.0 wt. % means an amount of oil ranging from about 1.0 wt. % to about 10.0 wt. %, including 1.0 and 10.0 wt. %.

The compositions of the present invention also comprise at least one surfactant. Suitable surfactants include ionic and non-ionic surfactants. Ionic surfactants include cationic and anionic surfactants. The adjuvant compositions of the present invention can include one or more non-ionic surfactants (i.e., one non-ionic surfactant, or a mixture of two or more different non-ionic surfactants) or one or more ionic surfactants (i.e., one ionic surfactant, or a mixture of two or more ionic surfactants), or a mixture of one or more non-ionic surfactants and one or more ionic surfactants.

Non-ionic surfactants are uncharged amphiphilic compounds. Non-limiting examples of suitable non-ionic surfactants include polyoxyethylene fatty acid esters, polyoxyethylene alkyl ethers (including ethers of fatty alcohols), polyoxyethylene sorbitan esters, polyoxyethylene glyceryl mono- and diesters, glyceryl mono- and distearate, sucrose distearate, propylene glycol stearate, long chain acyl hexosamides, long chain acyl amino acid amides, long chain acyl amides, glyceryl mono- and diesters, dimethyl acyl amines, C₁₂-C₂₂ fatty alcohols, C₁₂-C₂₂ glycol monoesters, and mixtures thereof.

In one embodiment, non-ionic surfactants are mono ethers or esters of polyoxyethylene (also referred to as mono ethers or esters of polyethylene oxide). As used herein, “polyoxyethylene” includes homopolymers and copolymers of ethylene oxide, for example random, graft, and block copolymers of ethylene oxide and propylene oxide. In a specific embodiment, one or more of the surfactants are selected from any of the Brij™ surfactants (available from Sigma Aldrich), for example cetyl ethers of polyoxyethylene. In another specific embodiment, one or more of the surfactants are selected from any of the Tween™ surfactants (available from Uniqema), for example polyoxyethylene sorbitan monooleate. In another specific embodiment, one or more of the surfactants are selected from any of the SPAN™ surfactants (available from Sigma Aldrich), for example sorbitan monolaurate (SPAN™ 20), sorbitan monooleate (SPAN™ 80), sorbitan triolate (SPAN™ 85), or other sorbitan fatty acid esters. In yet another specific embodiment, the surfactant is a cetyl ether of polyoxyethylene.

Non-limiting examples of suitable cationic surfactants include fatty amine salts (e.g., quaternary ammonium salts of alkyl amines) such as C₁₂-C₂₂ alkyl pyridinium salts, C₁₂-C₂₂ alkyl trialkyl ammonium salts, and C₁₂-C₂₂ alkyl ammonium salts, e.g., coconut alkyl amine acetate, stearyl, amine acetate, lauryl trimethyl ammonium chloride, stearyl trimethyl ammonium chloride, cetyl trimethyl ammonium chloride, di-stearyl dimethyl ammonium chloride, alkylbenzyl dimethyl ammonium chloride, etc.

Non-limiting examples of suitable anionic surfactants include soaps (e.g., fatty acid salts) and detergents (e.g., alkyl sulfate salts, alkyl benzenesulfonate salts, alkyl sulfonate salts, alkyl phosphonate salts, etc.), for example sodium dodecylsulfate, sodium oleate, sodium palmitate, sodium myristate, sodium stearate, sodium di(2-ethylhexyl) sulfosuccinate, etc.

Suitable surfactants of the present invention are not limited to any particular HLB value. In one embodiment, the at least one surfactant of the present invention can have an HLB value of less than about 12.

In another embodiment, the compositions of the present invention comprise about 0.1-15.0 wt. % of one or more surfactants (i.e., the wt. % of the total amount of surfactant in the composition, based on the total weight of the composition). In another embodiment, the compositions of the present invention comprise about 0.1-14.5 wt. %, or about 0.1-14.0 wt. %, or about 0.1-13.5 wt. %, or about 0.1-13.0 wt. %, or about 0.1-12.5 wt. %, or about 0.1-12.0 wt. %, or about 0.1-11.5 wt. %, or about 0.1-11.0 wt. %, or about 0.1-10.5 wt. %, or about 0.1-10.0 wt. %, or about 0.5-7.0 wt. %, or about 1.0-7.0 wt. %, or about 2.0-7.0 wt. % of one or more surfactants. The term “about” in regard to the weight range of the surfactant component refers to both the upper and lower limit specified. Thus, about 2.0-7.0 wt % means an amount of surfactant ranging from about 2.0 wt. % to about 7.0 wt. %, including 2.0 and 7.0 wt. %.

The compositions of the present invention include an aqueous phase. As used herein, the term “aqueous phase” includes pure water, aqueous buffer solutions, aqueous salt solutions (e.g. ˜0.9 wt/v % NaCl_{aq}), etc. In one embodiment, the aqueous phase is a buffer solution. Any physiologically acceptable buffer may be used, such as phosphate, acetate, tris, bicarbonate, carbonate, or the like. In another embodiment, the pH of the aqueous phase will be between 4.0-9.0. In still another embodiment, the pH of the aqueous phase will be about 4.5-8.5. In yet another embodiment, the aqueous phase is a phosphate buffer. In still yet another embodiment, the aqueous phase is cold water.

The term “aluminum salts” includes any aluminum salt suitable for use as a immunological adjuvant. For example, suitable aluminum salts includes aluminum phosphate (AlPO₄) and aluminum hydroxide (Al(OH)₃), optionally in the form of a hydrated gel. Aluminum hydroxide includes poorly crystalline aluminum oxyhydroxide with the structure of the mineral boehmite. Aluminum phosphate includes amorphous aluminum hydroxyphosphate, which may include low levels of sulfate ions. The molar ratio between aluminum and phosphate in amorphous aluminum hydroxy phosphate, and the molar ratio between aluminum and hydroxide in the aluminum hydroxide can vary from the “ideal” empirical formula, and provide pI values of about 11 for aluminum hydroxide, and pI values ranging from 5-7 for aluminum phosphate. Any commercially available aluminum hydroxide and aluminum phosphate is suitable for use in the compositions of the present invention.

The term “aluminum salt micro-/nano-particles” refers to particles of aluminum salts, as described above, having a particle size ranging from about 0.1 μm to about 50 μm, or about 0.1-25 μm, or about 0.1-20 μm, or about 0.1-10 μm, or about 0.1-5 μm, or about 0.1-3 μm, or about 3 μm.

The aluminum salt nano-/micro-particles of the present invention are surface stabilized with surfactant. As described herein, the surface stabilized aluminum salt nano-/micro-particles may be prepared by reducing the size of relatively large aluminum salt particles, e.g., particles having an average particle size of about 50 μm, suspended in a solution containing one or more surfactants. After reduction in size, the resulting surface stabilized aluminum salt nano-/micro-particles have a significantly lower average particle size, e.g., about 3 μm.

In one embodiment, the compositions of the present invention comprise 0.05-3.0 wt. % of aluminum salt nano-/micro-particles (i.e., the wt % of the total amount of aluminum salt nano-/micro-particles in the composition, based on the total weight of the composition). In another embodiment, the compositions of the present invention comprise about 0.1-3.0 wt. %, or about 0.5-3.0 wt. %, or about 0.5-2.5 wt. %, or about 0.5-2.0 wt. %, or about 0.5-1.5 wt. %, or about 0.5-1.0 wt. %, or about 0.5 wt. % of aluminum salt nano-/micro-particles. In yet another embodiment, the compositions of the present invention comprise about 0.5 wt. % of aluminum phosphate microparticles.

The vesicles of the compositions of the present invention can have any known morphology. For example, the vesicles of the present invention can be unilamellar vesicles having a single bilayer of surfactant or paucilamellar lipid vesicles having about 2 to 10 bilayers arranged in the form of substantially spherical shells separated by aqueous layers, surrounding a large amorphous central cavity free of lipid bilayers, for example those described in U.S. Pat. No. 6,387,373 (herein incorporated by reference in its entirety for all purposes). The vesicles of the present invention have a central cavity, carrying either water-soluble materials or a water-immiscible oily solution, which can be used to encapsulate one or more antigens. The water-immiscible oily solution comprises materials which are both water immiscible and immiscible in the surfactant or surfactants used to form the bilayers. The water immiscible oily material found in the amorphous central cavity can include any of the oils described herein.

In one embodiment, the compositions of the present invention comprise water- or oil-filled vesicles, e.g., vesicles having their amorphous central cavities filled with a water-miscible oily solution. These vesicles may be formed using the techniques described in U.S. Pat. No. 4,911,928 or U.S. Pat. No. 5,160,669, both of which are herein incorporated by reference in their entirety for all purposes.

In another embodiment, the vesicles of the compositions of the present invention are paucilamellar lipid vesicles. In yet another embodiment, the vesicles of the compositions of the present invention are Novasomes®, for example those described in U.S. Pat. Nos. 5,629,021 and 6,387,373, both of which are herein incorporated by reference in their entirety for all purposes. In still another embodiment, the vesicles of the compositions of the present invention are niosomes.

In either case, a lipid phase is formed by blending one or more oil and one or more surfactants, along with any sterols or lipophilic materials (e.g., one or more antigens) to be encapsulated in surfactant bilayers, to form a homogeneous lipid phase.

For example, any water-immiscible oily material to be encapsulated in the vesicles can be blended into the already formed oil phase, forming a lipophilic phase. Oil-soluble or oil-suspendable antigens to be encapsulated within the vesicles are first dispersed in the oil. The term “dispersed” as used herein includes dissolution or forming a suspension or colloid to yield a flowable phase. Once a lipophilic phase is made, it is blended with an aqueous phase (e.g., water, saline, buffer, or any other aqueous solution which will be used to hydrate the surfactants), which may also contain an antigen, under shear mixing conditions. “Shear mixing conditions”, as used herein, means a shear equivalent to a relative flow of 5-50 m/s through a 1 mm orifice.

Alternatively, the vesicles may first be formed using any known technique to provide vesicles which have the amorphous central cavity filled with an aqueous solution, possibly with some oil included. After formation of the substantially aqueous-filled surfactant vesicles, they are mixed with a water immiscible material, e.g., an oil, for example a volatile oil, to be incorporated into the amorphous central cavity under intermediate mixing conditions. The term “intermediate mixing conditions” means mixing of the preformed vesicles and the water immiscible material at or near room temperature under gentle conditions such as vortexing or syringing. Although flow conditions which yield a shear similar to that used to form the lipid vesicles initially could be used, it is often not essential, and in some cases may be counterproductive. The amorphous central cavity of the lipid vesicles is then filled with the water immiscible material, displacing the aqueous solution. The water immiscible material may act as a carrier for materials which are soluble or dispersed in it. The surfactant vesicles are then separated from any excess oil, e.g., by centrifugation.

It will be recognized by the skilled artisan that the vesicle component of the compositions of the present invention can be formed upon emulsifying a suitable mixture of at least one oil, at least one surfactant, optionally at least one sterol, and an aqueous phase.

In one embodiment, the vesicles are Novasomes®. Novasomes® are paucilamellar non-phospholipid vesicles ranging from about 50 nm to about 5 comprising BRIJ™ 72, cholesterol, oleic acid and squalene. In another embodiment, the paucilamellar non-phospholipid vesicles range in size from about 100 nm to about 500 nm.

Paucilamellar lipid vesicles can act to stimulate the immune response several ways, as non-specific stimulators, as carriers for the antigen, as carriers of additional adjuvants, and combinations thereof. Paucilamellar lipid vesicles act as non-specific immune stimulators when, for example, an antigenic composition or vaccine is prepared by intermixing the antigen with the preformed vesicles such that the antigen remains extracellular to the vesicles (either bound or unbound). Also, by encapsulating an antigen within the central cavity of the vesicle or absorbing an antigen in on the surface, the vesicle can act both as an immune stimulator and a carrier for the antigen. Thus, the vesicles can act as carriers for the antigen as is described in U.S. Pat. Nos. 4,855,090; 4,895,452; 4,911,928; 4,917,951; 5,000,960; 5,032,457; 5,160,669; 5,234,767; 5,628,936; 5,256,422; 5,405,615; 5,643,600; 5,665,380; 5,474,848; 5,651,062; 5,260,065; 5,628,936 5,032,457 and 6,387,373 (all of which are herein incorporated by reference in their entirety for all purposes).

In one embodiment, the antigen is mixed with the adjuvant composition of the present invention, creating a composition in which at least one antigen is bound to a paucilamellar lipid vesicle. In another embodiment, the vesicles are prepared in admixture with at least one selected antigen. In another embodiment, the vesicles are comprised of non-ionic surfactants. In another embodiment, the vesicles are comprised of lipids that are not phospholipids. In another embodiment, the vesicles can serve to carry additional adjuvants within the central cavity, between the bilayers, attached to the surface of the vesicle, or as an admixture, regardless of entrapment or absorption. In one embodiment, said adjuvant is selected from the group consisting of Complete Freund's Adjuvant (CFA), Incomplete Freund's Adjuvant (IFA), alum, and MF59. In another embodiment, any of the above compositions is prepared in an oil-in-water emulsion. In another embodiment, the paucilamellar lipid vesicle is a Novasome®.

In a specific embodiment, the antigen is mixed with a composition comprising an emulsion and aluminum salt nano-/micro-particles surface stabilized with at least one surfactant, wherein the emulsion comprises at least one oil, a plurality of surfactant vesicles (e.g., Novasomes® and/or niosomes), optionally at least one sterol, and an aqueous phase. In a further specific embodiment, the antigen is mixed with a composition comprising paucilamellar lipid vesicles (e.g., Novasomes® and/or niosomes).

Non-ionic surfactant vesicles (niosomes) prepared from a non-ionic surfactant, cholesterol and dicetyl phosphate are known in the art and have been extensively used in the cosmetic industry (see, U.S. Pat. Nos. 4,830,857 and 5,041,283, which are herein incorporated by reference in their entirety for all purposes). Niosomes appear to be multilamellar surfactant structures. Relatively insoluble compounds, such as the chemotherapeutics, currently formulated in liposomes, could be delivered in these synthetic, nonionic surfactant vehicles (see, Uchegbu et al. (1998) Int. J. Pharm. 172, 33-70, herein incorporated by reference in its entirety for all purposes). These vesicles are capable of entrapping and retaining water soluble solutes, are osmotically active and can be formulated to release entrapped solute slowly. One of the methods for producing niosomes involves drying a lipid to a thin film from organic solvent, and then hydrating this film with the aqueous solvent of choice. The resulting multilamellar vesicles can be further processed by sonication, extrusion, or other treatments to optimize drug entrapment. Other methods, such as injection of lipids in water-miscible or water-immiscible solvents into an aqueous solution, detergent dialysis, or reverse-phase evaporation are also available. Niosomes can be prepared, for example, from the non-ionic surfactant of the SPAN™ series, e.g. sorbitan monolaurate (SPAN™ 20) that can be used as a substitute for phospholipids. The physical characteristics of the vesicles were found to be dependent on the method of production. In one embodiment, when the antigen is mixed with the niosome lipid vesicles the antigen is trapped in the lipid vesicle. In another embodiment, the antigen is absorbed to the surface of the niosome. In another embodiment, the niosome and the antigen are in admixture, regardless of entrapment or absorption. In another embodiment, the niosome can serve to carry additional adjuvants within the central cavity, attached to the surface of the vesicle, or as an admixture, regardless of entrapment or absorption. In a further embodiment, said adjuvant is selected from the group consisting of Complete Freund's Adjuvant (CFA), Incomplete Freund's Adjuvant (IFA), alum, and MF59. In another embodiment, any of the above compositions is prepared in an oil-in-water emulsion. Niosomes can be used as an alternative to or to compliment liposomes and Novasomes®.

The compositions of the present invention optionally include at least one sterol. By “optionally” or “optional” we mean that the adjuvant compositions of the present invention may contain no sterol, or may contain one or more sterols. Non-limiting examples of sterols include, e.g. cholesterol, cholesterol derivatives, hydrocortisone, phytosterol, and mixtures thereof.

The amount of sterol can be adjusted depending upon the desired properties of the emulsion and vesicles. For example, the compositions of the present invention can comprise about 0-10.0 wt. %, or about 0.1-10.0 wt. %, or about 0.1-9.0 wt. %, or about 0.1-8.0 wt. %, or about 0.1-7.0 wt. %, or about 0.1-6.0 wt. %, or about 0.1-5.0 wt. %, or about 0.1-4.0 wt. %, or about 0.1-3.0 wt. %, or about 0.1-2.0 wt. %, or about 0.5-5.0 wt. %, or about 0.5-4.0 wt. %, or about 0.5-3.0 wt. %, or about 1.0 wt. %, or about 1.5 wt. %, or about 2.0 wt. % of sterol (i.e., the wt. % of the total amount of sterol in the composition, based on the total weight of the composition). The term “about” in regard to the weight range of the sterol component refers to both the upper and lower limit specified. Thus, about 0.5-3.0 wt. % means an amount of oil ranging from about 0.5 wt. % to about 3.0 wt. %, including 0.5 and 3.0 wt. %.

The compositions of the present invention can also include one or more ingredients capable of inducing positive charge. Non-limiting examples of ingredients capable of inducing positive charge include e.g., polycationic carbohydrates such as but not limited to inorganic or organic salts of chitosan and modified forms of chitosan (especially more positively charged ones), polyaminoacids such as polylysine, polyquaternary compounds, protamine, polyimine, DEAE-imine, polyvinylpyridine, polythiodiethylaminomethylethylene (P(TDAE)), polyhistidine, DEAE-methacrylate, DEAE-acrylamide, poly-p-aminostyrene, polyoxethane, co-polymethacrylates (e.g. copolymers of HPMA, N-(2-hydroxypropyl)-methacrylamide), GAFQUAT (U.S. Pat. No. 3,910,862; herein incorporated by reference in its entirety for all purposes), polyamidoamines, cetyl pyridinium chloride, stearylamine, diethanolamine, etc. In one embodiment, the ingredient capable of inducing positive charge is protamine.

The compositions of the present invention can also include an ingredient capable of inducing negative charge. Non-limiting examples of ingredients capable of inducing negative charge include e.g., oleic acid, palmitic acid, dicetyl phosphate, cetyl sulphate, phosphatidic acid, phosphatidyl serine, or mixtures thereof.

The amount of charge modifying compound ranges from about 0.01 to about 1.0 wt. %. In another embodiment, the amount of charge modifying compound ranges from about 0.01 to about 0.5 wt. %. In yet another embodiment, the amount of charge modifying compound ranges from about 0.1 to about 0.25 wt. %.

In another embodiment, the compositions of the present invention further comprise an immunologically effective amount of at least one antigen. An “antigen” is any substance which contains one or more epitopes, and when introduced into an animal or human, results in the initiation of a humoral and/or cell-mediated immune response. In particular, an antigen will stimulate a host's immune system to elicit an antigen-specific cellular immune response when the antigen is presented to an antigen presenting cell, a humoral antibody response, and/or an innate immune response. Normally, an epitope will include between about 3-15, generally about 5-15, amino acids. For purposes of the present invention, antigens can be derived from any virus, bacteria, parasite, fungi or tumor (see below). Furthermore, for purposes of the present invention, an “antigen” refers to a protein which may include modifications, such as deletions, additions and substitutions (generally conservative in nature), to the native sequence, as long as the protein maintains the ability to elicit an immunological response. These modifications may be deliberate, as through site-directed mutagenesis, or may be accidental, such as through mutations of hosts which produce the antigens.

An “immunological response” to an antigen or composition is the development in a subject of a humoral and/or a cellular immune response to molecules present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic or cytotoxic T-cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes or the lysis of cells infected with such microbes. Another aspect of cellular: immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

An “immunologically effective amount” or “pharmaceutically effective amount” of at least one antigen is a nontoxic but sufficient amount of one or more antigen which is sufficient to provide a clinically useful immune response in a patient. For example, if an antigen is intended to confer immunity against influenza, an immunologically effective amount of that antigen is the amount required to prevent an influenza infection, or reduce at least one symptom related to influenza virus infection in a patient. Alternatively, if the antigen is intended to induce antibody production in a mammal (e.g., for production of an antivenin), an immunologically effective amount of the antigen is the amount required to produce a clinically useful amount of antibody in the mammal. A clinically useful amount of antibody is, e.g. an amount of antibody which ameliorates symptoms in a patient or provides a measurable amount of antibody production in a patient. As will be pointed out below, the exact amount required will vary from subject to subject, depending on the species, age, the general condition of the subject, the severity of the condition being treated, the particular antigen of interest, the mode of administration and the like. An appropriate “effective” amount in any individual case may be determined by one of ordinary skill in the art using routine experimentation.

An antigenic composition or vaccine that elicits a cellular immune response may serve to sensitize a vertebrate subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

Thus, an immunological response as used herein may be one that stimulates the production of CTLs, and/or the production or activation of helper T-cells. The antigen of interest may also elicit an antibody-mediated immune response. Hence, an immunological response may also include one or more of the following effects: the production of antibodies by B-cells and/or the activation of suppressor T-cells. These responses may serve to neutralize infectivity, mediate antibody-complement, and/or antibody dependent cell cytotoxicity (ADCC) to provide protection to an immunized host. Such responses can be determined using standard immunoassays and neutralization assays, well known in the art.

An antigenic composition or vaccine which contains a selected antigen entrapped in, adsorbed to, or in admixture with a non-ionic surfactant vesicle, or a selected antigen combined with a composition comprising an emulsion and aluminum salt nano-/micro-particles surface stabilized with at least one surfactant displays “enhanced immunogenicity” when it possesses a greater capacity to elicit an immune response than the immune response elicited by an equivalent amount of the antigen without the vesicle or without the combination of emulsion and surface stabilized aluminum salt particles. Thus, a vaccine composition may display “enhanced immunogenicity” because the antigen is more strongly immunogenic or because a lower dose of antigen is necessary to achieve an immune response in the subject to which it is administered. Such enhanced immunogenicity can be determined by administering the non-ionic surfactant vesicle/antigen composition and controls to animals and comparing antibody titers or other immune response against the two using standard assays such as radioimmunoassay and ELISAs, well known in the art. In addition, the compositions of the invention can exhibit “enhanced immunogenicity” if said compositions can shift an immune response to the desired type of response. For example, the composition can be formulated to induce a “cellular response” or can be formulated to induce a “humoral response.”

The exact amount of antigen necessary will vary, depending on the subject being treated; the age and general condition of the subject to be treated; the capacity of the subject's immune system to synthesize antibodies; the degree of protection desired; the severity of the condition being treated; the particular antigen selected and its mode of administration, among other factors. An appropriate effective amount can be readily determined by one of skill in the art. Thus, a “therapeutically effective amount” will fall in a relatively broad range that can be determined through routine trials. For example, for purposes of the present invention, an effective dose will typically range from about 1 μg to about 100 mg, preferably from about 5 μg to about 3 mg, preferably from about 10 μg to about 1 mg and most preferably about 15 μg to about 500 μg of the antigen delivered per dose.

The term “patient” includes any animal, e.g. a mammal, in one embodiment a human.

As used herein, “treatment” refers to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection).

By “vertebrate subject” or “subject” is meant any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like are also non-limiting examples. Both adult and newborn individuals are intended to be covered.

The skilled artisan will recognize that the immunological effect of the compositions of the present invention can readily be determined by comparing the immunological effects of formulations, e.g. vaccines, comprising an antigen and a composition of the present invention with a formulation comprising the same antigen but lacking a composition of the present invention, a formulation comprising the same antigen but using a different adjuvant composition or a composition having no adjuvant.

The vaccine formulations comprising an adjuvant composition according to the invention may be suitable for protection or treatment of vertebrate subjects against a variety of disease states such as, for example, viral, bacterial, fungal or parasitic infections, cancer, allergies and autoimmune disorders. It is to be recognized that these specific disease states have been referred to by way of example only and are not intended to be limiting upon the scope of the present invention. The compositions of the invention are particularly useful for immunization against antigens which normally elicit poor immune responses. It is also useful for an antigen which does elicit a robust response because said composition would create an even greater response and thus can to avoid the need for a “booster” or additional immunizations.

Suitable antigens useful in combination with the compositions of the present invention include any antigen as defined herein. Antigens are commercially available or one of skill in the art is capable of producing them. The antigen can be either a modified-live or killed microorganism, or a natural product purified from a microorganism or other cell including, but not limited to, tumor cell, a synthetic product, a genetically engineered protein, peptide, polysaccharide or similar product, or an allergen. The antigenic moiety can also be a subunit of a protein, peptide, polysaccharide or similar product. The antigen may also be a genetic antigen, i.e., DNA or RNA that engenders an immune response.

Representative of the antigens that can be used according to the present invention include, but are not limited to, natural, recombinant or synthetic products derived from viruses, bacteria, fungi, parasites and other infectious agents in addition to autoimmune diseases, hormones, or tumor antigens which might be used in prophylactic or therapeutic vaccines and allergens. In one embodiment, the antigen comprises virus-like particles (VLPs) from various viruses such as influenza, HIV, RSV, Newcastle disease virus (NDV) etc. See PCT/US2006/40862, PCT/US2004/022001, U.S. Ser. No. 11/582,540, U.S. 60/799,343, U.S. 60/817,402, U.S. 60/859,240, all of which are herein incorporated by reference in their entirety. In another embodiment, the antigen comprises chimeric VLPs. “Chimeric VLPs” refer to VLPs that contain proteins, or portions thereof, from at least two different sources (organisms). Usually, one protein is derived from a virus that can drive the formation of VLPs from host cells. Thus, in one embodiment, said chimeric VLP comprises an RSV M protein. In another embodiment, said chimeric VLP comprises a NDV M protein. In another embodiment, said chimeric VLP comprises an influenza virus M protein.

The viral or bacterial products can be components which the organism produced by enzymatic cleavage or can be components of the organism that were produced by recombinant DNA techniques that are well known to those of ordinary skill in the art.

Some specific examples of antigens are antigens derived from viral infections caused by hepatitis viruses A, B, C, D & E3, human immunodeficiency virus (HIV), herpes viruses 1, 2, 6 & 7, cytomegalovirus, varicella zoster, papilloma virus, Epstein Barr virus, para-influenza viruses, adenoviruses, bunya viruses (e.g. hanta virus), coxsakie viruses, picoma viruses, rotaviruses, respiratory syncytial viruses, rhinoviruses, rubella virus, papovavirus, mumps virus, measles virus, polio virus (multiple types), adeno virus (multiple types), parainfluenza virus (multiple types), avian or pandemic influenza (various types), seasonal influenza, shipping fever virus, Western and Eastern equine encephalomyelitis, Japanese B. encephalomyelitis, Russian Spring Summer encephalomyelitis, hog cholera virus, Newcastle disease virus, fowl pox, rabies, feline and canine distemper and the like viruses, slow brain viruses, rous sarcoma virus (RSV), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, Japanese B encephalitis, Junin, Chikungunya, Rift Valley fever, yellow fever, meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia.

The antigens may also be derived from bacterial and fungal infections for example: antigens derived from infections caused by Mycobacteria causing TB and leprosy, pneumocci, aerobic gram negative bacilli, mycoplasma, staphyloccocal infections, streptococcal infections, salmonellae and chlamydiae, B. pertussis, Leptospira pomona, and icterohaemorrhagiae. Specific embodiments comprise S. paratyphi A and B, C. diphtheriae, C. tetani, C. botulinum, C. perfringens, C. feseri and other gas gangrene bacteria, B. anthracis, P. pestis, P. multocida, Neisseria meningitidis, N. gonorrheae, Hemophilus influenzae, Actinomyces (e.g., Norcardia), Acinetobacter, Bacillaceae (e.g., Bacillus anthrasis), Bacteroides (e.g., Bacteroides fragilis), Blastomycosis, Bordetella, Borrelia (e.g., Borrelia burgdorferi), Brucella, Candidia, Campylobacter, Chlamydia, Coccidioides, Corynebacterium (e.g., Corynebacterium diptheriae), Cryptococcus, Dermatocycoses, E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Meningiococcus, Pneumococcus and Streptococcus (e.g., Streptococcus pneumoniae and Groups A, B, and C Streptococci), Ureaplasmas, Treponema pollidum, and the like; Staphylococcus aureus, Plasmodium sp. (Pl. falciparum, Pl. vivax, etc.), Aspergillus sp., Candida albicans, Pasteurella haemolytica, Corynebacterium diptheriae toxoid, Meningococcal polysaccharide, Bordetella pertusis, Streptococcus pneumoniae (pneumococcus) polysaccharide, Clostridium tetani toxoid, Mycobacterium bovis, killed cells of Salmonella typhi, Cryptococcus neoformans, and Aspergillus.

The antigens may also be derived from parasitic malaria, leishmaniasis, trypanosomiasis, toxoplasmosis, schistosomiasis, filariasis malaria, Amebiasis, Babesiosis, Coccidiosis, Cryptosporidiosis, Dientamoebiasis, Dourine, Ectoparasitic, Giardias, Helminthiasis, Theileriasis, Trichomonas and Sporozoans (e.g., Plasmodium virax, Plasmodium fakiparium, Plasmodium malariae and Plasmodium ovale). These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), malaria, pregnancy complications, and toxoplasmosis.

Tumor-associated antigens suitable for use in compositions of the invention include both mutated and non-mutated molecules which may be indicative of single tumor type, shared among several types of tumors, and/or exclusively expressed or overexpressed in tumor cells in comparison with normal cells. In addition to proteins and glycoproteins, tumor-specific patterns of expression of carbohydrates, gangliosides, glycolipids and mucins have also been documented. Exemplary tumor-associated antigens for use in the subject cancer vaccines include protein products of oncogenes, tumor suppressor genes and other genes with mutations or rearrangements unique to tumor cells, reactivated embryonic gene products, oncofetal antigens, tissue-specific (but not tumor-specific) differentiation antigens, growth factor receptors, cell surface carbohydrate residues, foreign viral proteins and a number of other self proteins. Specific embodiments of tumor-associated antigens include, e.g., mutated antigens such as the protein products of the Ras p21 protooncogenes, tumor suppressor p53 and HER-2/neu and BCR-ab1 oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. Also included as tumor-associated antigens herein are whole cell and tumor cell lysates as well as immunogenic portions thereof, as well as immunoglobulin idiotypes expressed on monoclonal proliferations of B lymphocytes for use against B cell lymphomas. Tumor-associated antigens and their respective tumor cell targets include, e.g., cytokeratins, particularly cytokeratin 8, 18 and 19, as antigens for carcinoma. Epithelial membrane antigen (EMA), EphA1, EphA2, EphA3, EphA4, EphA5, EphA6, EphA7, EphA8, EphA10, EphB1, EphB2, EphB3, EphB4, EphB6, human embryonic antigen (HEA-125), human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. In cervical cancer, useful antigens could be encoded by human papilloma virus. Chromagranin-A and synaptophysin are antigens of neuroendocrine and neuroectodermal tumors. Of particular interest are aggressive tumors that form solid tumor masses having necrotic areas. The lysis of such necrotic cells is a rich source of antigens for antigen-presenting cells, and thus the subject therapy may find advantageous use in conjunction with conventional chemotherapy and/or radiation therapy. The antigens can be derived from any tumor or malignant cell line.

Antigens may also be derived from common allergens that cause allergies. Allergens include organic or inorganic materials derived from a variety of man-made or natural sources such as plant materials, metals, ingredients in cosmetics or detergents, latexes, or the like. Classes of suitable allergens for use in the compositions and methods of the invention can include, but are not limited to, pollens, animal dander, grasses, molds, dusts, antibiotics, stinging insect venoms, and a variety of environmental (including chemicals and metals) drug and food allergens. Common tree allergens include pollens from cottonwood, popular, ash, birch, maple, oak, elm, hickory, and pecan trees; common plant allergens include those from rye, ragweed, English plantain, sorrel-dock and pigweed; plant contact allergens include those from poison oak, poison ivy and nettles; common grass allergens include Timothy, Johnson, Bermuda, fescue and bluegrass allergens; common allergens can also be obtained from molds or fungi such as Alternaria, Fusarium, Hormodendrum, Aspergillus, Micropolyspora, Mucor and thermophilic actinomycetes; penicillin and tetracycline are common antibiotic allergens; epidermal allergens can be obtained from house or organic dusts (typically fungal in origin), from insects such as house mites (dermalphagoides pterosinyssis), or from animal sources such as feathers, and cat and dog dander; common food allergens include milk and cheese (diary), egg, wheat, nut (e.g., peanut), seafood (e.g., shellfish), pea, bean and gluten allergens; common environmental allergens include metals (nickel and gold), chemicals (formaldehyde, trinitrophenol and turpentine), Latex, rubber, fiber (cotton or wool), burlap, hair dye, cosmetic, detergent and perfume allergens; common drug allergens include local anesthetic and salicylate allergens; antibiotic allergens include penicillin and sulfonamide allergens; and common insect allergens include bee, wasp and ant venom, and cockroach calyx allergens. Particularly well characterized allergens include, but are not limited to, the major and cryptic epitopes of the Der pI allergen (Hoyne et al. (1994) Immunology 83, 190-195), bee venom phospholipase A2 (PLA) (Akdis et al. (1996) J. Clin. Invest. 98, 1676-1683), birch pollen allergen Bet v 1 (Bauer et al. (1997) Clin. Exp. Immunol. 107, 536-541), and the multi-epitopic recombinant grass allergen rKBG8.3 (Cao et al. (1997) Immunology 90, 46-51). These and other suitable allergens are commercially available and/or can be readily prepared as extracts following known techniques.

The antigen may be in the form of purified or partially purified antigen and can be derived from any of the above antigens, an antigenic peptide, proteins that are known and available in the art, and others that can identified using conventional techniques. The antigens will typically be in the form in which their toxic or virulent properties have been reduced or destroyed and which when introduced into a suitable host with the adjuvant of the invention, will either induce and immune response against the specific microorganisms, extract, or products of microorganisms used in the preparation of the antigen, or, in the case of allergens, they will aid in alleviating the symptoms of the allergy due to the specific allergen. The antigens can be used either singly or in combination; for example, multiple bacterial antigens, multiple viral antigens, multiple bacterial antigens, multiple parasitic antigens, multiple bacterial, viral toxoids, multiple tumor antigens, multiple allergens or combinations of any of the foregoing products can be combined with adjuvant compositions of the invention to create a polyvalent antigenic composition and/or a vaccine. In the compositions of the present invention, the antigen may be antigen entrapped in, adsorbed to, or in an admixture with the vesicle component of the composition.

In one embodiment, suitable antigens for use with the compositions of the present invention include antigens which are poorly immunogenic, for example malaria antigens, dengue antigens and HIV antigens, or antigens intended to confer immunity against pandemic diseases, for example influenza antigens.

The mechanism by which alum adjuvants enhance immune response is not fully understood. Alum adjuvants are thought to function by forming a depot at the site of injection, allowing for slow release of antigen and thus prolonging the time for interaction between antigen and antigen-presenting cells in lymphocytes. The adjuvant properties of alum may also be related to their ability to convert soluble antigens to particulate forms, which are more readily phagocytosed. However, alum adjuvants, alone, are ineffective in combination with certain antigens (typhoid vaccine, influenza hemagglutinin antigen, and Hib capsular polysaccharide-tetanus toxoid conjugate), perhaps because the immune response to these antigens does not depend on the release rate of the antigen, or because the antigens are not converted to particulate forms by alum.

Lipid vesicles induce both a humoral and cell-mediated immune response. Paucilamellar vesicles preferentially stimulate the Th1 sub-population of T-helper cells, and are effective with antigens of a broad size range, from short peptides to particulates. However, vesicles have the disadvantage of being osmotically sensitive, and can be incompatible with most organic solvents and some detergents. In addition, lipid vesicle adjuvants, alone, may be ineffective in combination with certain antigens.

Vaccines comprising a vesicle, e.g., a Novasome® or niosome, in combination with one or more suitable antigens (as described herein), and optionally other excipients (as described herein), can provide an improved immunogenic response compared to conventional vaccine compositions.

Emulsion type adjuvants such as Freund's Incomplete Adjuvant (an emulsion of mineral oil and mannide mononooleate) can cause significant side effects such as granulomas, inflammation at the inoculation site, and lesions, and are typically unstable upon freezing or two pH extremes. In addition, some emulsion type adjuvants (e.g., MF-59) are susceptible to oxidation in the presence of oxygen, peroxide, or metals. In addition, emulsion type adjuvants are not effective with all antigens. In addition, emulsion type adjuvants are less efficient than alum or vesicular adjuvants in holding the antigen at the injection site. For example, in most oil-in-water emulsion type adjuvants, the antigen is dispersed only in the aqueous phase of the emulsion, thereby not providing long-lasting depot action of the antigen.

For immunogenic compositions comprising an antigen admixed with a combination of alum and an emulsion containing a plurality of lipid vesicles, immune stimulation can occur by multiple mechanisms, thereby providing a synergistic immune stimulating effect. In addition, the alum and vesicle components act as depot-forming components, while the emulsion component fortifies immuno-stimulation. Thus, all three fractions (alum, vesicle, and emulsion) act in a complementary, synergistic manner. This combination can increase the antigenic response in a mammal inoculated with a vaccine comprising one or more antigens in combination with the adjuvant compositions of the present invention.

The synergistic effect provided by the combined action of alum and the emulsion containing a plurality of lipid vesicles is particularly important when used as an adjuvant for poorly immunogenic antigens, or antigens for pandemic diseases (e.g., influenza) in which it is highly desirable to maximize the number of clinically effective doses provided by a limited amount of antigen.

Vaccines comprising the adjuvant composition of the present invention provide clinically effective levels of immune response using substantially lower levels of alum compared to alum adjuvanted vaccines. For example, vaccines comprising the adjuvant compositions of the present invention comprise about 0.5-3.0 wt. % of aluminum salts, in one embodiment about 0.5 wt. % of aluminum salts, whereas conventional vaccines adjuvanted only with alum typically contain about 2.0-3.0 wt. % alum. Accordingly, vaccines comprising the adjuvant compositions of the present invention provide high levels of immune response while minimizing the undesirable side effects of alum adjuvants (localized reactions such as erythema, subcutaneous nodules, contact hypersensitivity, allergic reactions, granulomatous inflammation, slow degradation in vivo, etc.).

Similarly, the adjuvant compositions of the present invention provide for clinically effective levels of immune response while using substantially lower levels of the emulsion/vesicle components, thereby minimizing the undesirable side effects of emulsion type and paucilamellar adjuvants.

In one embodiment, the adjuvant compositions of the present invention comprise oil, alum, a non-ionic surfactant, a sterol, and an aqueous phase. The adjuvant composition is in the form of an emulsion comprising: (a) one or more oil, optionally one or more sterol, an aqueous phase, and one or more non-ionic surfactant components, (b) vesicles comprised of the oil, optional sterol, an aqueous phase, and non-ionic surfactant components, (c) and aluminum salt micro-/nano-particles surface stabilized with the non-ionic surfactant. The total amount of oil, aluminum salt micro-/nano-particles, non-ionic surfactant, and sterol is less than about 15 wt. %, or less than about 14 wt. %, or less than about 13 wt. %, or less than about 12 wt. %, or less than about 11 wt. %, or less than about 10 wt. %. In another embodiment, the total amount of oil, aluminum salt micro-/nano-particles, non-ionic surfactant, and sterol is about 10-15 wt. %, about 10-14 wt. %, about 10-13 wt. %, about 10-12 wt. %, or about 12 wt. %.

The compositions of the present invention are prepared by combining one or more oils, one or more non-ionic surfactants, optionally one or more sterols, aluminum salt particles, and an aqueous phase. The mixture is then subjected to shear mixing conditions, for example using a high-pressure homogenizer at a pressure of approximately 10,000 psi, and allowing the mixture to pass through the high-pressure homogenizer a sufficient number of times to provide an emulsion, vesicles, and aluminum salt particles of a reduced particle size (i.e., micro-/nano-particles), surface stabilized with the non-non-ionic surfactant. The aluminum salt particles prior to high-pressure homogenization can have any particle size which will not interfere with or clog the homogenizer, for example 50 μm particles. After homogenization, the particle size of the aluminum salt particles is reduced to an average particle size of typically <3 μm. In addition, since the particle size reduction takes place in the aqueous mixture of oil, non-ionic surfactant, and optional sterol, the non-ionic surfactant adsorbs to the surface of the smaller alum particles formed during homogenization, thereby providing surface-stabilized aluminum salt micro-/nano-particles.

In addition, the compositions of the invention can also be formulated with another adjuvant. Any adjuvant described in Vogel et al., “A Compendium of Vaccine Adjuvants and Excipients (2^{nd} Edition),” herein incorporated by reference in its entirety for all purposes, is envisioned within the scope of this invention.

The compositions of the present invention can also include one or more “pharmaceutically acceptable excipients or vehicles” such as water, saline, glycerol, polyethylene glycol, hyaluronic acid, ethanol, etc. Additionally, auxiliary substances, such as wetting or emulsifying agents, pH buffering substances, and the like, may be present in such vehicles.

The compositions of the present invention are useful as immunological adjuvants, and therefore, when combined with a suitable antigen, can elicit an immunological response when administered to a patient. In one embodiment, the immunological response elicited is an immune response which confers at least partial immunity in a patient to a pathogenic organism.

Vaccines comprising the compositions of the present invention can be prepared by combining one or more antigens with the composition of the present invention. By “combining”, we mean that the antigen can be combined with the components of the compositions of the present invention before, during, or after the formation of the composition of the present invention. For example, the antigen can be added to an already-formed composition of the present invention, or added to the raw materials of the composition of the present invention at any time during the formation of the composition—e.g., adding one or more antigens to a mixture of one or more oils, one or more surfactants, aluminum salt particles, the aqueous phase, and optionally one or more sterols, then emulsifying the oil and aqueous phases to form an emulsion, a plurality of vesicles, and aluminum salt nano-/micro-particles surface stabilized with the surfactant components; or alternatively adding one or more antigens at any time during the emulsification of the components of the compositions of the present invention.

Methods of administering vaccines comprising the compositions of the present invention include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions of the present invention are administered intramuscularly, intravenously, subcutaneously, transdermally or intradermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of the compositions of the present invention may induce an antibody or other immune response that is substantially higher than other routes of administration.

Administration can be systemic or local. In addition, the compositions of the present invention can be administered simultaneously with, just prior to, or subsequent to, another antigenic composition.

In yet another embodiment, a vaccine comprising the compositions of the present invention can be administered in such a manner as to target mucosal tissues in order to elicit an immune response at the site of immunization. For example, mucosal tissues such as gut associated lymphoid tissue (GALT) can be targeted for immunization by using oral administration of compositions which contain adjuvants with particular mucosal targeting properties. Additional mucosal tissues can also be targeted, such as nasopharyngeal lymphoid tissue (NALT) and bronchial-associated lymphoid tissue (BALT).

Dosage treatment may be a single dose schedule or a multiple dose schedule. A multiple dose schedule is one in which a primary course of administration and/or vaccinations may be with 1-10 separate doses, followed by other doses given at subsequent time intervals, chosen to maintain and/or reinforce the immune response, for example at 1-4 months for a second dose, and if needed, a subsequent dose(s) after several months. The boost may be with the compositions of the invention given for the primary immune response, or may be with a different formulation that contains the antigen. The dosage regimen will also, at least in part, be determined by the need of the subject and be dependent on the judgment of the practitioner. Furthermore, if prevention of disease is desired, the antigenic composition and/or vaccines are generally administered prior to primary infection with the pathogen of interest. If treatment is desired, e.g., the reduction of symptoms or recurrences, the vaccines are generally administered subsequent to primary infection.

In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose. In another embodiment, a second dose is administered about one month after the first administration and a third dose is administered about six months after the first administration. In yet another embodiment, the second dose is administered about six months after the first administration.

The vaccines of the present invention will comprise a “therapeutically or immunologically effective amount” of the antigen of interest. That is, an amount of antigen will be included in the compositions which, when in combination with the non-ionic surfactant vesicles, will cause the subject to produce a sufficient immunological response in order to prevent, reduce or eliminate symptoms. The exact amount necessary will vary, depending on the subject being treated; the age and general condition of the subject to be treated; the capacity of the subject's immune system to synthesize antibodies; the degree of protection desired; the severity of the condition being treated; the particular antigen selected and its mode of administration, among other factors. An appropriate effective amount can be readily determined by one of skill in the art. Thus, a “therapeutically effective amount” will fall in a relatively broad range that can be determined through routine trials. For example, for purposes of the present invention, an effective dose will typically range from about 1 μg to about 100 mg, preferably from about 5 μg to about 3 mg, preferably from about 10 μg to about 1 mg and most preferably about 15 μg to about 500 μg of the antigen delivered per dose.

Because vaccines comprising the compositions of the present invention provide enhanced cellular immune response in a patient, typically the number of administrations of vaccine, or the frequency of administration of vaccine can be reduced compared to conventional vaccines comprising the same antigen and a conventional adjuvant (e.g., alum only). Alternatively, the compositions of the present invention allow for reduced amounts of antigen, which can “conserve” or effectively increase the immunologically effective amount of antigens which are available in limited quantities. For example, the required amount of influenza antigen in vaccines comprising the compositions of the present invention can be reduced at least 50% compared to the amount of native antigen which provides the same level of immune response. In the case of poorly immunogenic antigens (e.g., HIV), vaccines comprising the compositions of the present invention can provide substantially enhanced immunogenic response compared to the native antigen.

In another embodiment, vaccine comprising the composition of the present invention can be administered as part of a combination therapy, for example, formulated with other immunogenic compositions and/or antivirals (e.g. Amantadine, Rimantadine, Zanamivir and Osteltamivir).

The dosage of vaccine comprising the compositions of the present invention can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. Dosages can be determined from animal studies. A non-limiting list of animals used to study vaccines include the guinea pig, Syrian hamster, chinchilla, hedgehog, chicken, rat, mouse and ferret. Most animals are not natural hosts for specific infections, but can still serve in studies of various aspects of the disease. For example, any of the above animals can be dosed with a vaccine of the present invention, to study the efficacy of a vaccine, to determine the immune response induced, and/or to determine if any neutralizing antibodies have been produced. For example, many studies have been conducted in the mouse model because mice are small size and their low cost allows researchers to conduct studies on a larger scale. Nevertheless, the mouse's small size also increases the difficulty of readily observing the clinical signs of the disease.

There has been extensive use of ferrets for studying various aspects of human influenza viral infection and its course of action. The development of many of the contemporary concepts of immunity to the influenza virus would have been impossible without the use of the ferret. Ferrets have proven to be a good model for studying influenza for several reasons: influenza infection in the ferret closely resembles that in humans with respect to clinical signs, pathogenesis, and immunity, types A and B of human influenza virus naturally infect the ferret, thus providing an opportunity to study a completely controlled population in which to observe the interplay of transmission of infection, illness, and sequence variation of amino acids in the glycoproteins of the influenza virus; and ferrets have other physical characteristics that make it an ideal model for deciphering the manifestations of the disease. For example, ferrets and humans show very similar clinical signs of influenza infection that seem to depend on the age of the host, the strain of the virus, environmental conditions, the degree of secondary bacterial infection, and many other variables. Thus, one skilled in the art can more easily correlate the efficacy of an influenza vaccine and dosage regiments from a ferret model to humans as compared to a mouse or any other model described above.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

### EXAMPLES

### Example 1

| | |
|---|---|
| Aluminum phosphate | 0.5% w/w |
| Soybean oil | 4.0% |
| BRIJ ™ 52 (Polyoxyethylene-2-cetyl ether) | 0.5% w/w |
| Saline | 95% w/w |

BRIJ™ 52 (0.5 g) was dissolved in soybean oil by heating at 50-60° C. The resulting solution was mechanically mixed with saline solution using a paddle-type mixer operated at 100-500 rpm and aluminum phosphate (commercially available, powder form, non-milled). The resulting mixture was fed into high-pressure homogenizer, such that the entire solution was passed through the homogenizer three times at a pressure of approximately 10,000 psi. The resulting adjuvant composition comprised an emulsion; alum micro-/nano-particles having a particle size range of 50 nm to 5 μm, with a mean particle size in the range of 100-150 nm; and surfactant vesicles.

### Example 2

| | |
|---|---|
| Aluminum phosphate | 0.5% w/w |
| Soybean oil | 4.0% w/w |
| BRIJ ™ 52 (Polyoxyethylene-2-cetyl ether) | 5.0% w/w |
| Cholesterol | 1.5% w/w |
| Phosphate buffered saline (pH 7.4) | 95% w/w |

Procedure: Dissolve BRIJ™ 52 and cholesterol in soybean oil by heating at 50-60° C. Mix with buffer and add aluminum phosphate while maintaining mixing using a mechanical mixer. Feed the mix into high-pressure homogenizer and allow three passes at 10,000 psi pressure.

Those skilled in the art will recognize, or be able to ascertain using no more than routine experimentation, many equivalents to the specific embodiments of the invention described herein. Such equivalents are intended to be encompassed by the following claims.

Any cited patents and publications referred to in this application are herein incorporated by reference in their entirety for all purposes. In addition, U.S. provisional applications 60/775,346 and 60/861,245 are herein incorporated by reference in their entirety for all purposes.

What is claimed is:

1. A composition comprising an emulsion and aluminum salt nano-/micro-particles surface stabilized with at least one surfactant, wherein the emulsion comprises:
at least one oil;
the at least one surfactant;
a plurality of surfactant vesicles;
optionally at least one sterol; and
an aqueous phase.

2. The composition of claim 1, further comprising an immunologically effective amount of at least one antigen.

3. The composition of claim 1, wherein the total weight percent of oil, surfactant, aluminum salt nano-/micro-particles and sterol is at most 15 wt. %.

4. The composition of claim 3, wherein the total weight percent of oil, surfactant, aluminum salt nano-/micro-particles, and sterol is approximately 12 wt. %.

5. The composition of claim 1, wherein at least one sterol is present in the composition.

6. The composition of claim 5, wherein the at least one sterol comprises cholesterol.

7. The composition of claim 1, further comprising at least one compound capable of inducing a positive charge.

8. The composition of claim 7, wherein the at least one compound capable of inducing a positive charge is protamine.

9. The composition of claim 1, wherein the pharmaceutical composition further comprises at least one compound capable of inducing a negative charge.

10. The composition of claim 9, wherein the at least one compound capable of inducing a negative charge is oleic acid.

11. The composition of claim 1, wherein the at least one oil comprises at least one oil selected from immunogenic oils.

12. The composition of claim 11, wherein the at least one oil comprises at least one oil selected from the group consisting of squalane, squalene, and mixtures thereof.

13. The composition of claim 1, wherein the at least one oil is selected from non-immunogenic oils.

14. The composition of claim 13, wherein the at least one oil comprises at least one oil selected from the group consisting of a vegetable oil, peanut oil, a fish oil, a lard oil, a mineral oil, vitamin E, and mixtures thereof.

15. The composition of claim 14, wherein the at least one oil comprises at least one oil selected from the group consisting of vegetable oils, nut oils, fish oils, lard oil, mineral oils, water insoluble vitamins, vitamin E, almond oil (sweet), apricot seed oil, borage oil, canola oil, coconut oil, corn oil, cotton seed oil, fish oil, jojoba bean oil, lard oil, linseed oil (boiled), Macadamia nut oil, triglyceride oil such as medium chain triglycerides, mineral oil, petrolatum, olive oil, peanut oil, safflower oil, sesame oil, soybean oil, squalene, squalane, sunflower seed oil, tricaprylin (1,2,3-trioctanoyl glycerol), wheat germ oil, rapeseed oil, avocado oil, flavor oils, and mixtures thereof.

16. The composition of claim 1, wherein the at least one surfactant is selected from non-ionic surfactants.

17. The composition of claim 16, wherein the at least one surfactant comprises at least one polyoxyethylene surfactant.

18. The composition of claim 17, wherein the at least one surfactant comprises a mono ether of polyethylene oxide.

19. The composition of claim 18, wherein the at least one surfactant comprises a polyoxyethylene-2-cetyl ether.

20. The composition of claim 1, wherein the total amount of the at least one oil is 0.5-40.0 wt. %.

21. The composition of claim 1, wherein the total amount of the at least one surfactant is 0.1-12.0 wt. %.

22. The composition of claim 1, wherein the total amount of the aluminum salt nano-/micro-particles is 0.05-3 wt. %.

23. The composition of claim 1, wherein the total amount of the aqueous phase is 60.0-98.0 wt. %.

24. The composition of claim 1, wherein the total amount of the at least one sterol is 0.1-10.0 wt. %.

25. The composition of claim 7, wherein the total amount of the at least one compound capable of inducing a positive charge is 0.01-1.0 wt. %.

26. The composition of claim 9, wherein the total amount of the at least one compound capable of inducing a negative charge is 0.01-1.0 wt. %.

27. The composition of claim 2, comprising:
0.5-40.0 wt. % of the at least one oil;
0.1-12.0 wt. % of the at least one surfactant;
0.05-3.0 wt. % of the aluminum salt nano-/micro-particles surface stabilized with the at least one surfactant;
0.1-10.0 wt. % of the at least one sterol; and
60.0-98.0 wt. % of the aqueous phase.

28. The composition of claim 27, wherein the total weight percent of oil, surfactant, aluminum salt nano-/micro-particles, and sterol is at most 15 wt. %.

29. The composition of claim 2, comprising:
3.0-6.0 wt. % of the at least one oil;
2.0-7.0 wt. % of the at least one surfactant;
0.5-3.0 wt. % of the aluminum salt nano-/micro-particles surface stabilized with the at least one surfactant;
0.5-3.0 wt. % of the at least one sterol; and
8.0-90.0 wt. % of the aqueous phase.

30. The composition of claim 29, wherein the total weight percent of oil, surfactant, aluminum salt nano-/micro-particles, and sterol is approximately 12 wt. %.

31. The composition of claim 2, comprising:
about 4.0 wt. % of the at least one oil;
about 5.0 wt. % of the at least one surfactant;
about 0.5 wt. % of the aluminum salt nano-/micro-particles surface stabilized with the at least one surfactant;
about 1.5 wt. % of the at least one sterol; and
about 88 wt. % of the aqueous phase.

32. The composition of claim 1, wherein the aluminum salt nano-/micro-particles have an average particle size of <3 μM.

33. The composition of claim 1, wherein the at least one oil, a least one surfactant, and an aqueous phase form an oil-in-water emulsion.

34. A composition prepared by a process comprising:
(a) combining at least one oil, an aqueous phase, aluminum salt particles, at least one surfactant, and optionally at least one sterol; and
(b) mixing the combination of step (a) under shear mixing conditions, whereby the at least one oil, the aqueous phase, at least one surfactant, and optionally at least one sterol form an emulsion and a plurality of surfactant vesicles, and the aluminum salt particles are reduced in size to form aluminum salt nano-/micro-particles surface stabilized with at least one surfactant.

35. The composition of claim 34, prepared by a process further comprising:
(c) adding an immunologically effective amount of at least one antigen.

36. A process comprising:
(a) combining at least one oil, an aqueous phase, aluminum salt particles, at least one surfactant, and optionally at least one sterol; and
(b) mixing the combination of step (a) under shear mixing conditions, whereby the at least one oil, an aqueous phase, at least one surfactant, and optionally at least one sterol form an emulsion and a plurality of surfactant vesicles, and the aluminum salt particles are reduced in size to form aluminum salt nano-/micro-particles surface stabilized with at least one surfactant.

37. The process of claim 36, further comprising:
(c) adding an immunologically effective amount of at least one antigen.

38. The process of claim 36, wherein said combining comprises combining:
0.5-40.0 wt. % of the at least one oil;
0.1-12.0 wt. % of the at least one surfactant;
0.05-3.0 wt. % of the aluminum salt particles;
0.1-10.0 wt. % of the at least one sterol; and
60.0-98.0 wt. % of the aqueous phase.

38. A vaccine comprising the composition of claim 2.

39. A method of eliciting a cellular immune response in a patient comprising administering to the patient a therapeutically effective amount of a composition of claim 2.

40. A method of immunizing which comprises administering to a patient in need thereof a therapeutically effective amount of a composition of claim 2.
